(19) United States
(12) Patent Application Publication
(10) Pub. No.: US 2021/0186365 A1
(43) Pub. Date: Jun. 24, 2021

(54) METHODS FOR DETERMINING A POSITION OF A FIRST MEDICAL DEVICE WITH RESPECT TO A SECOND MEDICAL DEVICE, AND RELATED SYSTEMS AND MEDICAL DEVICES

(71) Applicant: Baylis Medical Company Inc., Montreal (CA)

(72) Inventors: Steven Kinio, Mississauga (CA); Gareth Davies, Toronto (CA)

(21) Appl. No.: 16/720,190

(22) Filed: Dec. 19, 2019

(51) Int. Cl. A61B 5/06; A61M 25/06; A61B 90/00

(52) U.S. Cl. CPC A61B 5/065; A61M 25/0612; G01R 27/2605; A61B 2090/0811; A61B 90/08

(57) ABSTRACT

A first medical device includes an elongate member having a lumen extending longitudinally therethrough from a proximal end to a distal end. A second medical device includes a needle advanceable through the lumen from the proximal end towards the distal end. A capacitor is configured so that the capacitance of the capacitor is a measure of a longitudinal position of the needle with respect to the elongate member. A capacitance sensor is configured to sense the capacitance of the capacitor as the needle is advanced through the lumen. A processor is in communication with the capacitance sensor. An output device is in communication with the processor and is configured to generate an output indicative of the longitudinal position of the needle with respect to the elongate member.

### FIELD

This document relates to medical devices. More specifically, this document relates to methods for determining a position of a first medical device with respect to a second medical device, and related systems and medical devices.

### SUMMARY

The following summary is intended to introduce the reader to various aspects of the detailed description, but not to define or delimit any invention.

Systems of medical devices are disclosed. According to some aspects, a system of medical devices includes first medical device having an elongate member. The elongate member has a proximal portion defining a proximal end, a distal portion defining a distal end, and a lumen extending longitudinally therethrough from the proximal end to the distal end. The system further includes a second medical device including a needle advanceable through the lumen from the proximal end towards the distal end. The system further includes a capacitor including a first electrical conductor supported by the elongate member, and a second electrical conductor supported by one of the elongate member and the second medical device. The first electrical conductor and second electrical conductor are spaced apart and electrically insulated from each other. As the needle is advanced through the lumen, a capacitance of the capacitor is a measure of a longitudinal position of the needle with respect to the elongate member. The system further includes a capacitance sensor electrically connectable to the first electrical conductor and the second electrical conductor and configured to sense the capacitance of the capacitor as the needle is advanced through the lumen, and to generate a sensor signal based on the sensed capacitance. The system further includes a processor in communication with the capacitance sensor and configured to receive and process the sensor signal from the capacitance sensor, and generate a processor signal based on the sensor signal. The system further includes an output device in communication with the processor and configured to receive the processor signal and generate an output based on the processor signal. The output is an indicator of the longitudinal position of the needle with respect to the elongate member.

In some examples, the first electrical conductor and second electrical conductor are supported by the elongate member. The elongate member can include a sidewall extending longitudinally between the proximal end and the distal end, and radially between an inner surface and an outer surface, and the first electrical conductor can include a first plate embedded in the sidewall on a first side of the lumen. The second electrical conductor can include a second plate embedded in the sidewall on a second side of the lumen opposite the first side. At least one of the first electrical conductor and the second electrical conductor can extend continuously from the proximal portion to the distal portion. Alternatively, at least one of the first electrical conductor and the second electrical conductor can be positioned within the distal portion, for example proximate the distal end.

In some examples, the system includes a second capacitor. The second capacitor can include a third electrical conductor including a third plate, and a fourth electrical conductor including a fourth plate. The third plate and fourth plate can be spaced apart and electrically insulated from each other. As the needle is advanced through the lumen, a capacitance of the second capacitor can be an additional measure of a longitudinal position of the needle with respect to the elongate member. The third plate can be embedded in the sidewall on the first side of the lumen, and spaced proximally from the first plate. The fourth plate can be embedded in the sidewall on the second side of the lumen, spaced proximally from the second plate.

In some examples, the first electrical conductor is supported by the elongate member, and the second electrical conductor is supported by the second medical device. In some examples, the needle is the second electrical conductor. In some such examples, the elongate member can include a sidewall extending longitudinally between the proximal end and the distal end, and radially between an inner surface and an outer surface. The first electrical conductor can be embedded in the sidewall and can extend continuously from the proximal portion to the distal portion.

In some examples, the system further includes a first shield electrode supported by the elongate member and spaced radially outwardly and electrically insulated from the first electrical conductor. The first shield electrode can be electrically connectable to the capacitance sensor and configured to shield the first electrical conductor from parasitic capacitance. In some examples the system further includes a second shield electrode supported by the elongate member and spaced radially outwardly and electrically insulated from the second electrical conductor. The second shield electrode can be electrically connectable to the capacitance sensor and configured to shield the second electrical conductor from parasitic capacitance.

In some examples, the output device includes a light. The output can include illumination of the light when the needle is at a predetermined longitudinal position with respect to the elongate member. In some examples, the output device includes a screen. The output can include a GUI showing an image of the longitudinal position of the needle with respect to the elongate member.

Medical devices are also disclosed. According to some aspects, a medical device includes an elongate member having a proximal portion defining a proximal end, a distal portion defining a distal end, a sidewall extending longitudinally between the proximal end and the distal end and radially between an inner surface that and an outer surface, and a lumen defined by the inner surface and extending longitudinally through the elongate member from the proximal end to the distal end. The medical device further includes a capacitor including a first electrical conductor supported by the sidewall and a second electrical conductor spaced apart and electrically insulated from the first electrical conductor and supported by the sidewall. A first electrical connector is electrically connected to the first electrical conductor and electrically connectable to a capacitance sensor. A second electrical connector is electrically connected to the second electrical conductor and electrically connectable to the capacitance sensor.

In some examples, the first electrical conductor includes a first plate embedded in the sidewall on a first side of the lumen, and the second electrical conductor includes a second plate embedded in the sidewall on a second side of the lumen opposite the first side. The first plate and the second plate can extend continuously from the proximal portion to the distal portion.

In some examples, the first electrical conductor is longitudinally spaced from the second electrical conductor.

In some examples, the first electrical conductor includes a first plate embedded in the sidewall on a first side of the lumen, and the second electrical conductor includes a second plate embedded in the sidewall on a second side of the lumen opposite the first side. The first plate and the second plate can be positioned within the distal portion, for example proximate the distal end.

In some examples, the medical device includes a second capacitor. The second capacitor can include a third electrical conductor including a third plate, and a fourth electrical conductor including a fourth plate. The third plate and fourth plate can be spaced apart and electrically insulated from each other. The third plate can be embedded in the sidewall on the first side of the lumen, spaced proximally from the first plate, and the fourth plate can be embedded in the sidewall on the second side of the lumen, spaced proximally from the second plate.

In some examples, the capacitor further includes a first shield electrode associated with the elongate member and spaced radially outwardly and electrically insulated from the first electrical conductor. The first shield electrode can be configured to shield the first electrical conductor from parasitic capacitance.

In some examples, the capacitor further includes a second shield electrode associated with the elongate member and spaced radially outwardly and electrically insulated from the second electrical conductor. The second shield electrode can be configured to shield the second electrical conductor from parasitic capacitance.

Methods for determining a position of a first medical device with respect to a second medical device are also disclosed. According to some aspects, a method includes: a. advancing a second medical device into a lumen of a first medical device, from a proximal end of the first medial device towards a distal end of the first medical device; b. during step a., sensing a capacitance of a capacitor associated with the first medical device and the second medical device, whereby the capacitance is a measure of a longitudinal position of the second medical device with respect to the first medical device; and c. generating an output based on the capacitance, wherein the output is an indicator of the longitudinal position of the second medical device with respect to the first medical device.

In some examples, step a. includes passing the second medical device through a gap between a first electrical conductor and second electrical conductor of the capacitor.

In some examples, step a. includes, while advancing the second medical device, advancing a second electrical conductor of the capacitor with respect to a first electrical conductor of the capacitor.

In some examples, step a. includes passing the second medical device past the capacitor.

In some examples, step c. includes generating the output when the capacitance reaches a predetermined value.

In some examples, step c. includes generating an image of the longitudinal position of the needle with respect to the elongate member and updating the image as the capacitance changes.

In some examples, the method further includes adjusting a position of the second medical device with respect to the first medical device based on the output.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of a first example system of medical devices, showing a first medical device, second medical device, and control unit, spaced apart and disconnected from each other;

FIG. 2 is a perspective view of the system of FIG. 1, showing the second medical device inserted into the first medical device, and the first medical device connected to the control unit;

FIG. 3 is a longitudinal cross section taken along line 3-3 in FIG. 2, schematically showing the control unit and related electrical connections;

FIG. 4 is a transverse cross section taken along line 4-4 in FIG. 2;

FIG. 5 is a simplified graph showing the change in sensor signal as the second medical device of FIGS. 1 to 4 is advanced through the first medical device of FIGS. 1 to 4;

FIG. 6 is a cross-section similar to that of FIG. 3, showing a first medical device, second medical device, control unit, and electrical connections of a second example system;

FIG. 7 is a simplified graph showing the change in sensor signal as the second medical device of FIG. 6 is advanced through the first medical device of FIG. 6;

FIG. 8 is a cross-section similar to that of FIG. 3, showing a first medical device, second medical device, control unit, and electrical connections of a third example system;

FIG. 9 is a cross-section similar to that of FIG. 4, taken through the first electrical conductor of the first medical device and the second medical device of the third example system;

FIG. 10 is a simplified graph showing the change in sensor signal as the second medical device of FIGS. 8 and 9 is advanced through the first medical device of FIGS. 8 and 9;

FIG. 11 is a perspective view of a fourth example system of medical devices, showing a first medical device, second medical device, and control unit, spaced apart and disconnected from each other;

FIG. 12 is a longitudinal cross section similar to that of FIG. 3, schematically showing the control unit and related electrical connections;

FIG. 13 is a transverse cross section similar to that of FIG. 4; and

FIG. 14 is a cross-section similar to that of FIG. 3, showing a first medical device, second medical device, control unit, and electrical connections of a fifth example system.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are systems of medical devices that include one or more capacitors, and one or more capacitance sensors. For example, a system of medical devices can include a first medical device (e.g. a catheter in the form of a sheath or dilator) that in use is advanced into a patient's body to a target location (e.g. advanced via a femoral vein to a patient's heart) and a second medical device (e.g. a perforation device including a needle) that in use is passed through the first medical device towards the target location. A capacitor can be associated with the first medical device and the second medical device, and a capacitance sensor can be connected to the capacitor, to sense the capacitance of the capacitor as the second medical device is advanced through the first medical device. The capacitor can be configured so that the capacitance of the capacitor changes as the second medical device is advanced through the first medical device. The capacitance can thus be used as a measure of a position of the second medical device with respect to the first medical device.

For example, the capacitor can include a pair of electrical conductors that are supported by the first medical device, so that as the second medical device is advanced through the first medical device, the second medical device passes through a gap between the pair of electrical conductors. For further example, the capacitor can include a pair of electrical conductors that are supported by the first medical device, so that as the second medical device is advanced through the first medical device, the second medical device passes beside (but not between) the pair of electrical conductors. For further example, the capacitor can include a pair of electrical conductors, where one of the electrical conductors is supported by the first medical device, and one of the electrical conductors is supported by the second medical device, so that as the second medical device is advanced through the first medical device, the relative position of the electrical conductors changes.

In the above examples, as the second medical device is advanced through the first medical device, the capacitance of the capacitor changes, and by sensing the capacitance of the capacitor, the relative position of the first medical device and second medical device can be determined. For example, by sensing the capacitance, it can be determined whether a perforating tip of a needle is approaching a distal end of the catheter, or has passed beyond a distal end of the catheter. This can facilitate ease of use of the medical devices, and enhance patient safety.

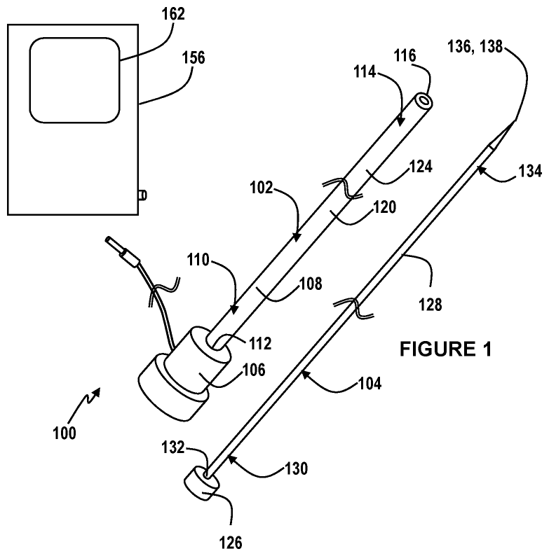

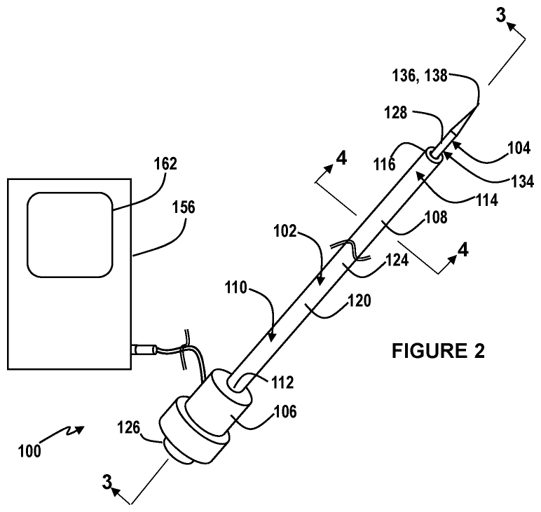

Referring now to FIGS. 1 and 2, a first example system 100 of medical devices is shown. In the example shown, the system 100 includes a first medical device 102 in the form of a catheter, and a second medical device 104 in the form of a perforation device. The catheter can be, for example, a sheath, a dilator, or an alternative device that is intended for use by passing another medical device therethrough (e.g. coaxially therethrough). The perforation device can be, for example, a mechanical perforation device, or a radiofrequency (RF) perforation device. In alternative examples, the second medical device can be alternative type of medical device that is intended for use by being passed through another medical device.

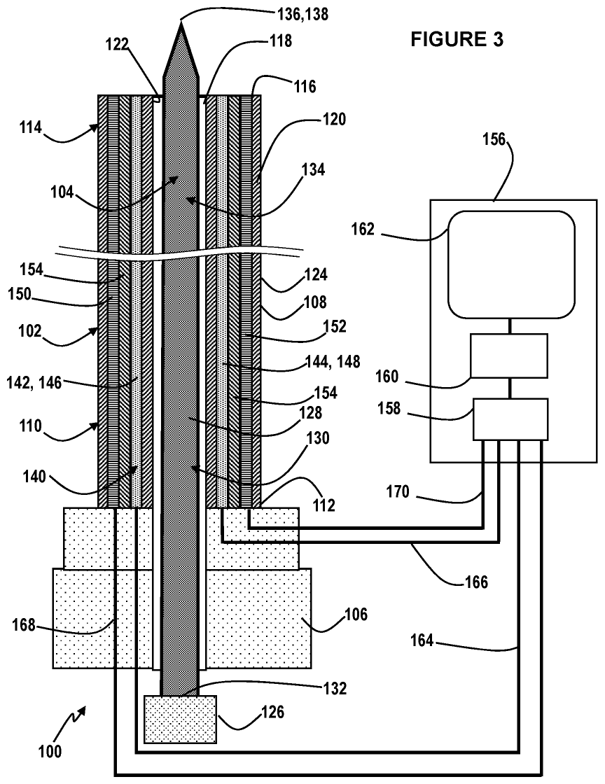

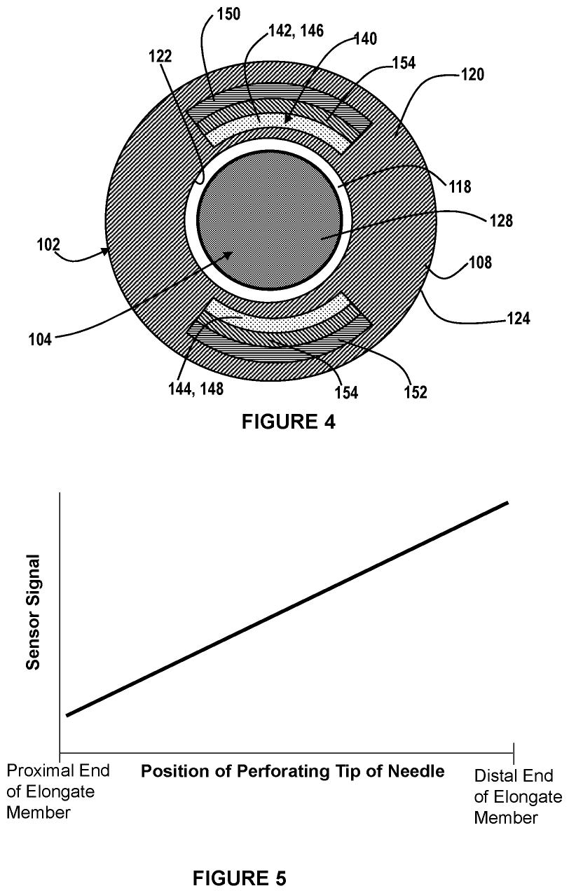

Referring also to FIGS. 3 and 4, in the example shown, the first medical device 102 includes a hub 106 and an elongate member 108 extending from the hub 106. The elongate member 108 has a proximal portion 110 defining a proximal end 112 of the elongate member 108, a distal portion 114 defining a distal end 116 of the elongate member 108, and a lumen 118 (shown in FIGS. 3 and 4) extending longitudinally through the elongate member 108 from the proximal end 112 to the distal end 116. The elongate member 108 includes a sidewall 120, which extends longitudinally between the proximal end 112 and the distal end 116, and radially between an inner surface 122 (shown in FIGS. 3 and 4) that defines the lumen 118, and an outer surface 124.

Referring still to FIGS. 1 to 4, in the example shown, the second medical device 104 includes a hub 126 and a needle 128 extending from the hub 126. The needle 128 has a proximal portion 130 defining a proximal end 132 of the needle 128, and a distal portion 134 defining a distal end 136 of the needle 128. The distal end 136 of the needle 128 includes a perforating tip 138. As shown in FIG. 2, the needle 128 is advanceable through the hub 106 and through the lumen 118, from the proximal end 112 of the elongate member 108 towards the distal portion 114 of the elongate member 108, to position the perforating tip 138 of the needle 128 proud of the distal end 116 of the elongate member 108.

Referring to FIGS. 3 and 4, the system further includes a capacitor 140, which is associated with the first medical device 102 and the second medical device 104. As used herein, the term “associated with” indicates that the first medical device 102, second medical device 104, and capacitor 140 are configured so that the capacitance of the capacitor 140 changes as a function of the position of the second medical device 104 with respect to the first medical device 102, as will be described below.

Referring still to FIGS. 3 and 4, the capacitor 140 includes a first electrical conductor 142, and a second electrical conductor 144. In the example shown, the first electrical conductor 142 and the second electrical conductor 144 are both supported by the elongate member 108. In alternative examples (as will be described below), the first electrical conductor can be supported by the elongate member, and the second electrical conductor can be supported by the second medical device. That is, in general, the first electrical conductor can be supported by the elongate member, and the second electrical conductor can be supported by the elongate member or the second medical device (i.e. supported by one of the elongate member and the second medical device). As used herein, the term “supported by” indicates that the referenced electrical conductor is integral with, embedded in, connected to, mounted to, adhered to, affixed to, or otherwise secured to the referenced part, so that the referenced electrical conductor moves with the referenced part.

Referring still to FIGS. 3 and 4, the first electrical conductor 142 and second electrical conductor 144 are spaced apart and are electrically insulated from each other, to form the capacitor 140. More specifically, the first electrical conductor 142 is in the form of a first plate 146, which is embedded in the sidewall 120 on a first side of the lumen 118. The second electrical conductor 144 is in the form of a second plate 148, which is embedded in the sidewall 120 on a second side of the lumen 118 opposite the first side. The first electrical conductor 142 and second electrical conductor 144 are spaced apart by a portion of the sidewall 120, and by the lumen 118.

The first plate 146 and second plate 148 can be, for example, a metal such as copper. The first plate 146 and second plate 148 can optionally be in the form of a tape. The first plate 146 and the second plate 148 can be, for example, up to about 3 mm in width, and about 0.035 mm or more in thickness. In alternative examples, the first plate and second plate can be of another size (e.g. only nanometers thick), provided that they are not in electrical contact. The first plate 146 and the second plate 148 can optionally be curved, as shown in FIG. 4, to match the curve of the sidewall 120.

In the example shown, the first plate 146 and second plate 148 are each embedded in the sidewall 120, and each extend continuously along the entire length of the elongate member 108, from the proximal portion 110 to the distal portion 114. In alternative examples, the first plate and second plate can extend along less than the entire length of the elongate member, such as along a majority of the length of the elongate member, or along only a small section of the elongate member. For example, the first plate and the second plate can be relatively short in length (e.g. about 5 mm), and can be positioned within the distal portion, proximate the distal end (e.g. right at the distal end, or slightly proximal of the distal end).

Referring still to FIGS. 3 and 4, in the example shown, the system 100 further includes first 150 and second 152 shield electrodes, which are supported by the elongate member 108. The first 150 and second 152 shield electrodes are spaced radially outwardly from the first 142 and second 144 electrical conductors, respectively, and are electrically insulated from the first 142 and second 144 electrical conductors, respectively, by electrical insulation 154. In the example shown, the electrical insulation 154 is a strip of insulative material; however, in alternative examples, electrical insulation could be provided by the material of the sidewall. As will be described in further detail below, the first 150 and second 152 shield electrodes can shield the first 142 and second 144 electrical conductors, respectively, from parasitic capacitance.

Referring still to FIGS. 3 and 4, the system further includes a control unit 156. The control unit 156 houses a capacitance sensor 158, a processor 160, and an output device 162, described in further detail below.

Referring still to FIGS. 3 and 4, the capacitance sensor 158 can be, for example, a commercially available capacitance sensor such as the one sold by Texas Instruments Incorporated under model number FDC1004. In the example shown, the capacitance sensor 158 is electrically connectable to the first electrical conductor 142 and the second electrical conductor 144, as well as to the first shield electrode 150 and the second shield electrode 152. Specifically, in the example shown, the system includes a first electrical connector 164 (e.g. a wire) that is electrically connected to the first electrical conductor 142, and extends from the first electrical conductor 142 for electrical connection with the capacitance sensor 158 (e.g. for connection with a capacitive sense port of the capacitance sensor 158), a second electrical connector 166 (e.g. a wire) electrically connected to the second electrical conductor 144 and extending from the second electrical conductor 144 for electrical connection with the capacitance sensor 158 (e.g. for connection with a ground port of the capacitance sensor), a third electrical connector 168 (e.g. a wire) electrically connected to the first shield electrode 150 and extending from the first shield electrode 150 for electrical connection with the capacitance sensor 158 (e.g. for connection with a first shield port of the capacitance sensor), and a fourth electrical connector 170 (e.g. a wire) electrically connected to the second shield electrode 152 and extending from the second shield electrode 152 for electrical connection with the capacitance sensor 158 (e.g. to a second shield port of the capacitance sensor).

The first through fourth electrical connectors 164-170 can optionally be removably electrically connectable to the capacitance sensor 158, for example via a common male connector (shown in FIG. 1) that connects to a female connector of the control unit 156.

The capacitance sensor 158 can sense the capacitance of the capacitor 140 as the needle 128 is advanced through the lumen 118. More specifically, in the example shown, in use, the capacitance sensor 158 can continuously sense the capacitance of the capacitor 140 as the needle 128 is advanced through the lumen 118. Furthermore, the capacitance sensor 158 can activate the shield electrodes 150, 152 to shield the capacitor 140 from the effects of parasitic capacitance (e.g. due to other instruments in the vicinity of the elongate member 108).

As shown in FIG. 5, as the needle 128 is advanced into the lumen 118, the capacitance of the capacitor 140 will change, due to the effect of the needle 128 on the dielectric permittivity between the first electrical conductor 142 and the second electrical conductor 144. The capacitance can thus be used as a measure of the longitudinal position of the needle 128 with respect to the elongate member 108. For example, the capacitance (e.g. the value of the capacitance, or a trend in the capacitance, or a change in the capacitance) can be an indicator of when the perforating tip 138 is well shrouded within the elongate member 108 (and thus when a patient's anatomy is protected from being perforated by the needle 128), or when the perforating tip 138 is at the distal end 116 of the elongate member 108 (i.e. when the needle 128 is ‘primed’ for use), or when the perforating tip 138 has passed beyond the distal end 116 of the elongate member 108 and is exposed (and thus when a patient's anatomy is not protected from being perforated by the needle 128).

Referring back to FIG. 3, the capacitance sensor 158 is in communication with the processor 160, and the processor 160 is in communication with an output device 162. The capacitance sensor 158 can sense the capacitance of the capacitor 140 as the needle 128 is advanced through the lumen 118, and can generate a signal (referred to herein as a “sensor signal”) based on the sensed capacitance. The processor 160 is configured to receive and process the signal from the capacitance sensor 158, and to generate a signal (referred to herein as a “processor signal”) based on the sensor signal. The output device 162 can receive the processor signal, and can generate an output based on the processor signal.

In some examples, the output device 162 can include a light, and the processor signal can cause illumination of the light, or change in color of the light. For example, when the sensor signal indicates that the capacitance has reached a predetermined value that corresponds to the perforating tip 138 being at a predetermined longitudinal position with respect to the elongate member 108 (e.g. the perforating tip being at the distal end 116 of the elongate member 108), the processor can signal the output device 162 to change the color of the light from green to red.

In some examples, the output device 162 can include a screen that shows a graphical user interface (GUI). The processor signal can cause the output device 162 to generate an image showing the longitudinal position of the needle 128 within the elongate member 108. For example, as the needle 128 is advanced through lumen 118 and the capacitance changes, the image can change based on the capacitance.

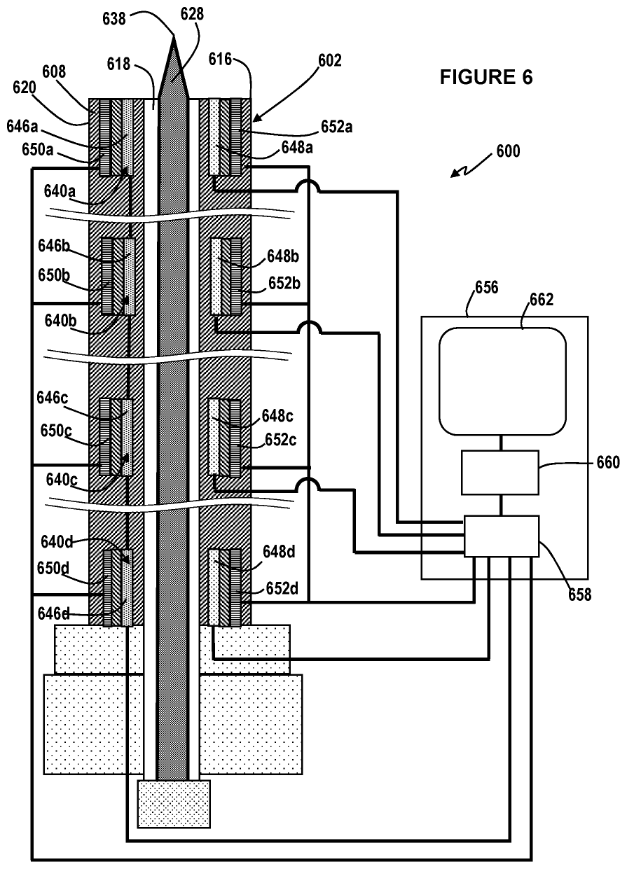

Referring now to FIG. 6, an alternative example system is shown. In the example of FIG. 6, features that are like those of FIGS. 1 to 5 will be referred to with like reference numerals, incremented by 500.

In the example of FIG. 6, the first medical 602 device includes several discrete capacitors (i.e. a first capacitor 640a, a second capacitor 640b, a third capacitor 640c, and a fourth capacitor 640d), spaced along the length of the elongate member 608. Each capacitor includes a pair of electrical conductors in the form of plates, which are embedded in the sidewall 620 on opposed sides of the lumen 618, and are spaced apart and electrically insulated from each other. That is, the first capacitor 640a includes a first plate 646a and a second plate 648a, the second capacitor 640b includes a third plate 646b and a fourth plate 648b, which are spaced proximally from the first plate 646a and second plate 648a, the third capacitor 640c includes a fifth plate 646c and a sixth plate 648c, which are spaced proximally from the third plate 646b and fourth plate 648b, and the fourth 640d capacitor includes a seventh plate 646d and an eighth plate 648d, which are spaced proximally from the fifth plate 646c and sixth plate 648c. Each plate is electrically connectable to the capacitance sensor 658. For example, the first 646a, third 646b, fifth 646c, and seventh 646d plates can be connected to a common ground port of the capacitance sensor 658, and the second 648a, fourth 648b, sixth 648c, and eighth 648d plates can be connected to a respective capacitance sense port of the capacitance sensor 658.

Referring still to FIG. 6, the system further includes a set of shield electrodes 650a-d and 652a-d, each of which is associated with one of the plates 646a-d and 648a-d, respectively, and each of which is connected to the capacitance sensor 658 (e.g. via shield ports).

Similarly to the example of FIGS. 1 to 5, the system 600 includes a control unit 656, which houses the capacitance sensor 658, a processor 660 that is in communication with the capacitance sensor 658 and configured to receive and process sensor signals from the capacitance sensor 658 and generate processor signals based on the sensor signals, and an output device 662 in communication with the processor 660 and configured to receive the processor signals and generate an output based on the processor signals.

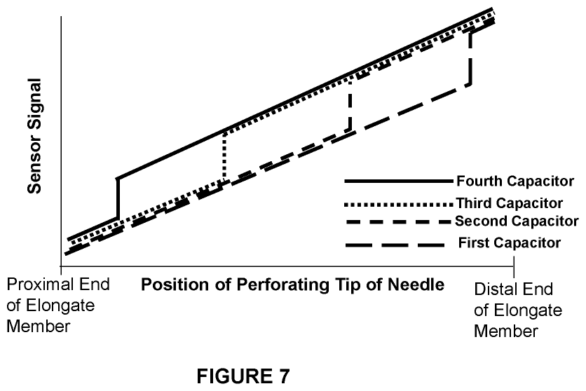

Referring also to FIG. 7, as the needle 628 is advanced into the lumen 618, the capacitance of each capacitor 640a-d will change as the needle 628 passes by that capacitor. The capacitance can thus be used as a measure of the longitudinal position of the needle 628 with respect to the elongate member 608. As noted above, the capacitance can be an indicator of when the perforating tip 638 is well shrouded within the elongate member 608 (and thus when a patient's anatomy is protected from being perforated by the needle 628), or when it is at the distal end 616 of the elongate member 608 (i.e. when it is ‘primed’ for use), or when it has passed beyond the distal end 616 of the elongate member 608 and is exposed (and thus when a patient's anatomy is not protected from being perforated by the needle 628).

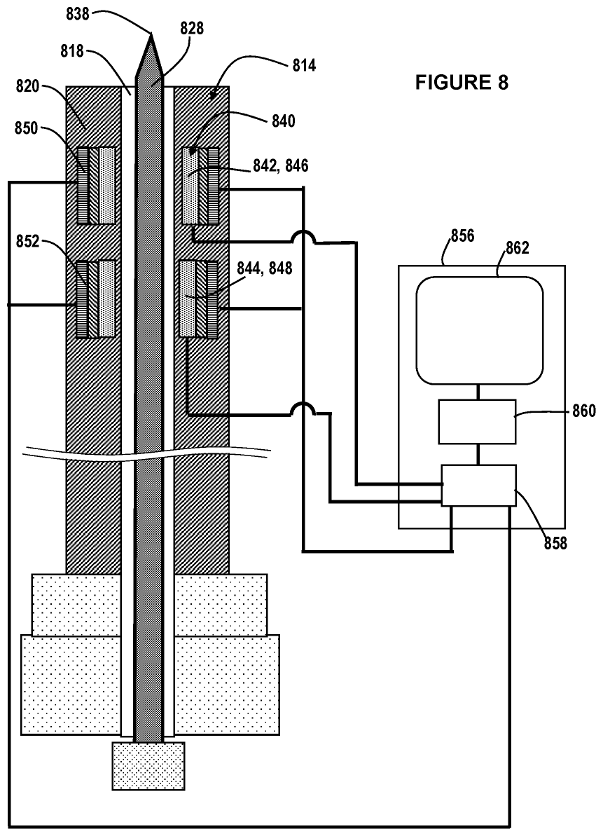

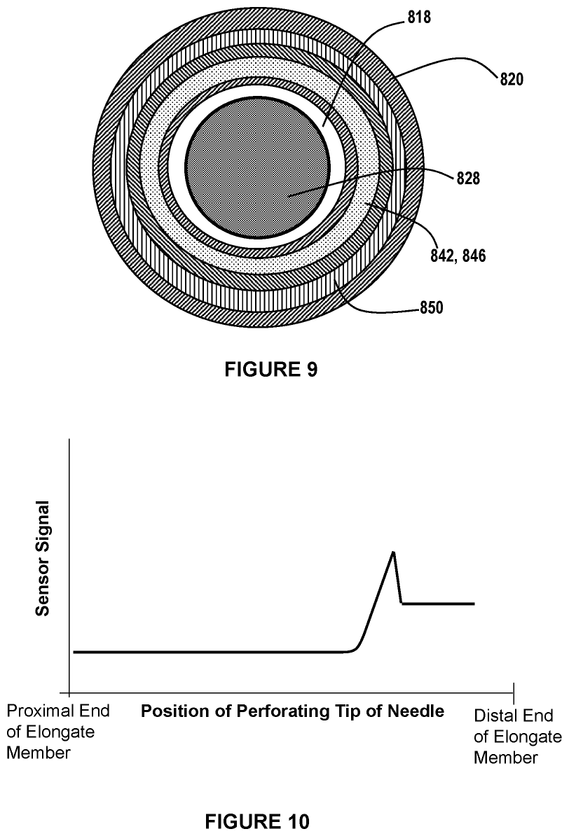

Referring now to FIGS. 8 to 10, an alternative example is shown. In the example of FIGS. 8 to 10, features that are like those of FIGS. 1 to 5 will be referred to with like reference numerals, incremented by 700.

In the example of FIGS. 8 to 10, the first 842 and second 844 electrical conductors of the capacitor 840 are in the form of a first ring 846 and a second ring 848, respectively. The first ring 846 and second ring 848 are embedded in the sidewall 820 in the distal portion 814, and are longitudinally spaced apart. The first ring 846 and second ring 848 are connected to the capacitance sensor 858 (e.g. to a capacitance sense port and a ground port of the capacitance sensor, respectively).

Similarly to the example of FIGS. 1 the 5, the system further includes shield electrodes 850, 852, each of which is associated with one of the electrical conductors 842, 844, respectively, and each of which is connected to the capacitance sensor 858.

Furthermore, similarly to the example of FIGS. 1 to 5, the system includes a control unit 856, which houses the capacitance sensor 858, a processor 860 that is in communication with the capacitance sensor 858 and configured to receive and process sensor signals from the capacitance sensor 858 and generate processor signals based on the sensor signals, and an output device 862 in communication with the processor 860 and configured to receive the processor signals and generate an output based on the processor signals.

In the example of FIGS. 8 to 10, as the needle 828 is advanced through the lumen 818, the needle 828 passes beside, but not between, the electrical conductors 842, 844 of the capacitor 840. As shown in FIG. 10, as the needle 828 passes beside the electrical conductors 842, 844, a spike is observed in the sensor signal. The spike indicates that the perforating tip 838 of the needle 828 is at the longitudinal position of the capacitor 840.

In alternative examples, rather than rings, the electrical conductors can be in the form of longitudinally spaced apart plates.

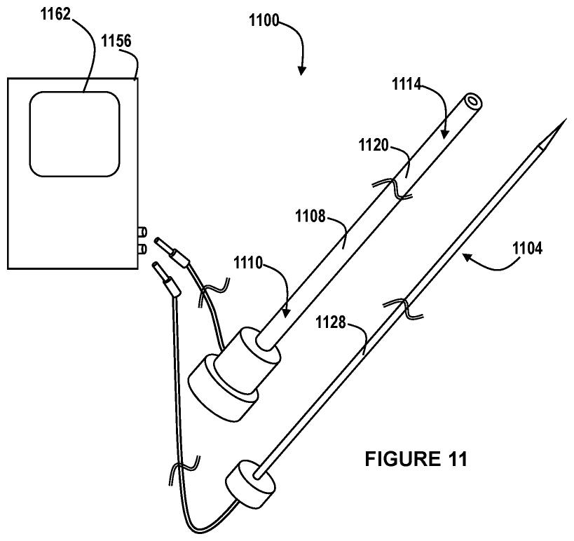

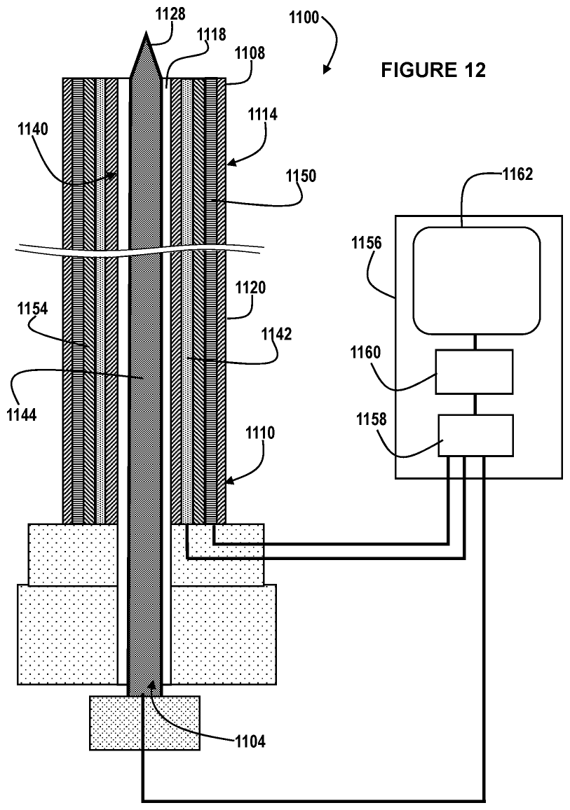

Referring now to FIGS. 11 to 13 a further alternative example is shown. In the example of FIGS. 11 to 13, features that are like those of FIGS. 1 to 5 will be referred to with like reference numerals, incremented by 1000.

In the example of FIGS. 11 to 13, the second electrical conductor 1144 of the capacitor 1140 is supported by the second medical device 1104. Particularly, in the example shown, the needle 1128 itself serves as the second electrical conductor 1144, and is electrically connected to the capacitance sensor 1158. The first electrical conductor 1142 is embedded in the sidewall 1120 of the elongate member 1108, is generally tubular, and extends from the proximal portion 1110 of the elongate member 1108 to the distal portion 1114 of the elongate member 1108.

In the example shown, the system 1100 further includes a shield electrode 1150, which is embedded in the sidewall 1120 of the elongate member 1108 radially outwardly of the first electrical conductor 1142, is generally tubular, and extends from the proximal portion 1110 of the elongate member 1108 to the distal portion 1114 of the elongate member 1108. Insulation 1154 is positioned between the first electrical conductor 1142 and the shield electrode 1150.

In the example shown, the needle 1128, the first electrical conductor 1142, and the shield electrode 1150 are electrically connectable to the capacitance sensor 1158. Similarly to the example of FIGS. 1 to 5, the system 1110 includes a control unit 1156, which houses the capacitance sensor 1158, a processor 1160 that is in communication with the capacitance sensor 1158 and configured to receive and process sensor signals from the capacitance sensor 1158 and generate processor signals based on the sensor signals, and an output device 1162 in communication with the processor 1160 and configured to receive the processor signals and generate an output based on the processor signals.

Similarly to the examples of FIGS. 1 to 5, as the needle 1128 is advanced through the lumen 1118, the capacitance of the capacitor 1140 will change as a function of the longitudinal position of the needle 1128, and can be used as a measure of the longitudinal position of the needle 1128 with respect to the elongate member 1108.

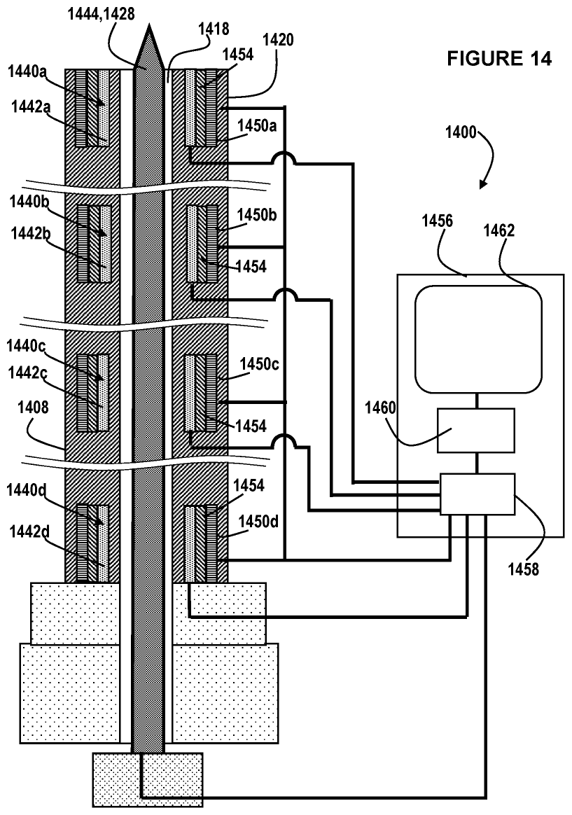

Referring now to FIG. 14, a further alternative example is shown. In the example of FIG. 14, features that are like those of FIGS. 1 to 5 will be referred to with like reference numerals, incremented by 1300.

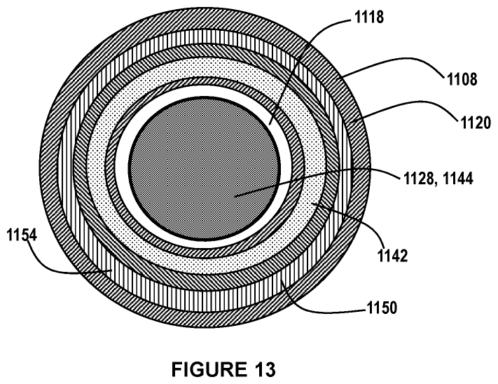

The example of FIG. 14, the system 1400 includes a set of capacitors, i.e. a first capacitor 1440a, a second capacitor 1440b, a third capacitor 1440c, and a fourth capacitor 1440d. Each capacitor 1440a-d includes a respective first electrical conductor 1442a-d in the form of a ring (not shown in transverse section, but similar to the transverse section shown in FIG. 13). The rings are longitudinally spaced apart and embedded in the sidewall 1420. The needle 1428 itself serves as the second electrical conductor 1444 of each capacitor 1440a-d.

In the example shown, the system 1400 further includes a respective shield electrode 1450a-d associated with each ring. The shield electrodes 1450a-d are embedded in the sidewall 1420 of the elongate member 1408, and spaced radially outwardly of each first electrical conductors 1442a-d, respectively. Insulation 1454 is positioned between each first electrical conductor 1442a-d and each shield electrode 1450a-d, respectively.

The needle 1428, the first electrical conductor 1442a-d of each respective capacitor 1440a-d, and the shield electrodes 1450a-d are electrically connectable to the capacitance sensor 1458. Similarly to the example of FIGS. 1 to 5, the system 1400 includes a control unit 1456, which houses the capacitance sensor 1458, a processor 1460 that is in communication with the capacitance sensor 1458 and configured to receive and process sensor signals from the capacitance sensor 1458 and generate processor signals based on the sensor signals, and an output device 1462 in communication with the processor 1460 and configured to receive the process or signals and generate an output based on the processor signals.

As the needle 1428 is advanced through the lumen 1418, the capacitance of each capacitor 1440a-d will change as a function of the longitudinal position of the needle 1428, in a similar fashion to that shown in FIG. 7, and can be used as a measure of the longitudinal position of the needle 1428 with respect to the elongate member 1408.

In any of the above examples, in order to reduce parasitic capacitance, “out-of-phase” sensing can be used. That is, the electrical conductor that is connected to the ground port of the capacitive sensor can be connected to a shield output of the electrical conductor that is connected to the capacitive sense port, to cancel out some sources of parasitic capacitance.

In any of the above examples, in order to mitigate parasitic capacitance, reference electrodes can be mounted in the elongate member, to normalize the first electrical conductor and/or second electrical conductor with reference capacitive signals.

The devices and systems described above can be used in various medical procedures, but may be particularly useful in transseptal perforation procedures, in which a dilator (i.e. a first medical device) is advanced via the femoral vein towards the heart and positioned adjacent the fossa ovalis of the atrial septum, and then a transseptal perforation device (i.e. a second medical device) is advanced into and through the lumen of the dilator, from the proximal end of the dilator towards the distal end of the dilator. In such procedures, as the transseptal perforation device is advanced through the lumen of the dilator, capacitance can be sensed, to provide a measure of the longitudinal position of the transseptal perforation device with respect to the dilator. That is, as described above, capacitance can be sensed as the transseptal perforation device is passed through a gap between a first electrical conductor and second electrical conductor of the capacitor (as shown in FIGS. 1 to 5 and 6 to 7), and/or capacitance can be sensed as a second electrical conductor of the capacitor is advanced with respect to a first electrical conductor of the capacitor (as shown in FIGS. 11 to 13, and 14), and/or capacitance can be sensed as the transseptal device passes past the capacitor (as shown in FIGS. 8 to 10).

As described above, an output can be generated based on the capacitance, to provide an indication of the longitudinal position of the transseptal perforation device with respect to the dilator. For example, an output can be generated when the capacitance reaches a predetermined value. The output can be, for example, in the form of an image, or a light. This can help an operator to ensure that the perorating tip of the perforation device is shrouded within the dilator until it is ready for use by the operator.

Optionally, based on the output, the position of the transseptal perforation device can be adjusted with respect to the dilator. For example, if a red light illuminates before the user is ready to perforate the fossa ovalis, the user can withdraw the transseptal perforation device proximally, until a green light illuminates.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A system of medical devices, comprising:
a first medical device comprising an elongate member, the elongate member having a proximal portion defining a proximal end, a distal portion defining a distal end, and a lumen extending longitudinally therethrough from the proximal end to the distal end;
a second medical device comprising a needle advanceable through the lumen from the proximal end towards the distal end;
a capacitor comprising a first electrical conductor supported by the elongate member, and a second electrical conductor supported by one of the elongate member and the second medical device, wherein the first electrical conductor and second electrical conductor are spaced apart and electrically insulated from each other, and whereby as the needle is advanced through the lumen, a capacitance of the capacitor is a measure of a longitudinal position of the needle with respect to the elongate member;
a capacitance sensor electrically connectable to the first electrical conductor and the second electrical conductor and configured to sense the capacitance of the capacitor as the needle is advanced through the lumen, and to generate a sensor signal based on the sensed capacitance;
a processor in communication with the capacitance sensor and configured to receive and process the sensor signal from the capacitance sensor, and generate a processor signal based on the sensor signal;
an output device in communication with the processor and configured to receive the processor signal and generate an output based on the processor signal, wherein the output is an indicator of the longitudinal position of the needle with respect to the elongate member.

2. The system of medical devices of claim 1, wherein the first electrical conductor and second electrical conductor are supported by the elongate member.

3. The system of medical device of claim 2, wherein the elongate member comprises a sidewall extending longitudinally between the proximal end and the distal end, and radially between an inner surface and an outer surface, and wherein the first electrical conductor comprises a first plate embedded in the sidewall on a first side of the lumen.

4. The system of medical devices of claim 3, wherein the second electrical conductor comprises a second plate embedded in the sidewall on a second side of the lumen opposite the first side.

5. The system of medical devices of claim 4, wherein at least one of the first electrical conductor and the second electrical conductor extends continuously from the proximal portion to the distal portion.

6. The system of medical devices of claim 4, wherein at least one of the first electrical conductor and the second electrical conductor is positioned within the distal portion, proximate the distal end.

7. The system of medical devices of claim 6, wherein:
the system further comprises a second capacitor, the second capacitor comprising third electrical conductor comprising a third plate, and a fourth electrical conductor comprising a fourth plate, wherein the third plate and fourth plate are spaced apart and electrically insulated from each other, whereby as the needle is advanced through the lumen, a capacitance of the second capacitor is an additional measure of a longitudinal position of the needle with respect to the elongate member;
the third plate is embedded in the sidewall on the first side of the lumen, spaced proximally from the first plate; and
the fourth plate is embedded in the sidewall on the second side of the lumen, spaced proximally from the second plate.

8. The system of medical device of claim 1, wherein the first electrical conductor is supported by the elongate member, and the second electrical conductor is supported by the second medical device.

9. The system of medical devices of claim 8, wherein the needle is the second electrical conductor.

10. The system of medical devices of claim 9, wherein the elongate member comprises a sidewall extending longitudinally between the proximal end and the distal end, and radially between an inner surface that and an outer surface, and wherein the first electrical conductor is embedded in the sidewall and extends continuously from the proximal portion to the distal portion.

11. The system of medical devices of claim 1, wherein the system further comprises a first shield electrode supported by the elongate member and spaced radially outwardly and electrically insulated from the first electrical conductor, the first shield electrode electrically connectable to the capacitance sensor and configured to shield the first electrical conductor from parasitic capacitance.

12. The system of medical devices of claim 11, wherein the system further comprises a second shield electrode supported by the elongate member and spaced radially outwardly and electrically insulated from the second electrical conductor, the second shield electrode electrically connectable to the capacitance sensor and configured to shield the second electrical conductor from parasitic capacitance.

13. The system of medical devices of claim 1, wherein the output device comprises at least one of:
a light, wherein the output comprises illumination of the light when the needle is at a predetermined longitudinal position with respect to the elongate member; and
a screen, wherein the output comprises a GUI showing an image of the longitudinal position of the needle with respect to the elongate member;

14. A medical device comprising:
an elongate member having a proximal portion defining a proximal end, a distal portion defining a distal end, a sidewall extending longitudinally between the proximal end and the distal end and radially between an inner surface that and an outer surface, and a lumen defined by the inner surface and extending longitudinally through the elongate member from the proximal end to the distal end;
a capacitor comprising a first electrical conductor supported by the sidewall and a second electrical conductor spaced apart and electrically insulated from the first electrical conductor and supported by the sidewall;
a first electrical connector electrically connected to the first electrical conductor and electrically connectable to a capacitance sensor; and
a second electrical connector electrically connected to the second electrical conductor and electrically connectable to the capacitance sensor.

15. The medical device of claim 14, wherein the first electrical conductor comprises a first plate embedded in the sidewall on a first side of the lumen, and the second electrical conductor comprises a second plate embedded in the sidewall on a second side of the lumen opposite the first side, and wherein the first plate and the second plate extend continuously from the proximal portion to the distal portion.

16. The medical device of claim 14, wherein the first electrical conductor is longitudinally spaced from the second electrical conductor.

17. The medical device of claim 14, wherein the first electrical conductor comprises a first plate embedded in the sidewall on a first side of the lumen, and the second electrical conductor comprises a second plate embedded in the sidewall on a second side of the lumen opposite the first side, wherein the first plate and the second plate are positioned within the distal portion, proximate the distal end.

18. The medical device of claim 17, further comprising a second capacitor, wherein the second capacitor comprises third electrical conductor comprising a third plate and a fourth electrical conductor comprising a fourth plate, the third plate and fourth plate are spaced apart and electrically insulated from each other, the third plate is embedded in the sidewall on the first side of the lumen, spaced proximally from the first plate, and the fourth plate is embedded in the sidewall on the second side of the lumen, spaced proximally from the second plate.

19. The medical device of claim 14, wherein the capacitor further comprises a first shield electrode associated with the elongate member and spaced radially outwardly and electrically insulated from the first electrical conductor, the first shield electrode configured to shield the first electrical conductor from parasitic capacitance.

20. The medical device of claim 19, wherein the capacitor further comprises a second shield electrode associated with the elongate member and spaced radially outwardly and electrically insulated from the second electrical conductor, the second shield electrode configured to shield the second electrical conductor from parasitic capacitance.

21. A method for determining a position of a first medical device with respect to a second medical device, the method comprising:
a. advancing the second medical device into a lumen of the first medical device, from a proximal end of the first medial device towards a distal end of the first medical device;
b. during step a. sensing a capacitance of a capacitor associated with the first medical device and the second medical device, whereby the capacitance is a measure of a longitudinal position of the second medical device with respect to the first medical device; and
c. generating an output based on the capacitance, wherein the output is an indicator of the longitudinal position of the second medical device with respect to the first medical device.

22. The method of claim 21, wherein step a. comprises passing the second medical device through a gap between a first electrical conductor and second electrical conductor of the capacitor.

23. The method of claim 21, wherein step a. comprises, while advancing the second medical device, advancing a second electrical conductor of the capacitor with respect to a first electrical conductor of the capacitor.

24. The method of claim 21, wherein step a. comprises passing the second medical device past the capacitor.

25. The method of claim 21, wherein step c. comprises generating the output when the capacitance reaches a predetermined value.

26. The method of claim 21, wherein step c. comprises generating an image of the longitudinal position of the needle with respect to the elongate member and updating the image as the capacitance changes.

27. The method of claim 21, further comprising adjusting a position of the second medical device with respect to the first medical device based on the output.
